(19) United States
(12) Patent Application Publication
(10) Pub. No.: US 2022/0265927 A1
(43) Pub. Date: Aug. 25, 2022

(54) SYSTEMS AND METHODS FOR TRANSITIONING FROM AUTOMATED INSULIN DELIVERY

(71) Applicant: Tandem Diabetes Care, Inc., San Diego, CA (US)

(72) Inventors: Paul Harris, San Diego, CA (US); Virginia S. Lu, San Diego, CA (US)

(21) Appl. No.: 17/677,621

(22) Filed: Feb. 22, 2022

Related U.S. Application Data
(60) Provisional application No. 63/152,154, filed on Feb. 22, 2021.

(51) Int. Cl. A61M 5/172; A61M 5/142; G16H 40/63; G16H 20/17

(52) U.S. Cl. CPC A61M 5/1723; A61M 5/14244; G16H 40/63; G16H 20/17; A61M 2230/201

(57) ABSTRACT

Disclosed herein are apparatuses and methods for transitioning from automated closed loop insulin delivery to open loop insulin therapy. When closed loop control is terminated, rather than immediately transitioning from the closed loop rate at termination to a preprogrammed open loop rate, the system can instead gradually transition over time to the preprogrammed open loop rate. This gradual transition provides a safer transition to open loop therapy that reduces the risk of hyperglycemia and hypoglycemia.

### RELATED APPLICATION

This application claims the benefit of U.S. Provisional Application No. 63/152,154 filed Feb. 22, 2021, which is hereby fully incorporated herein by reference.

### FIELD OF THE INVENTION

The present invention relates generally to ambulatory infusion pumps and, more particularly, to operation of ambulatory infusion pumps in a closed-loop or semi-closed-loop fashion.

### BACKGROUND OF THE INVENTION

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices that can provide continuous subcutaneous insulin injection and/or infusion therapy as an alternative to multiple daily insulin injections via syringe or injector pen. Such ambulatory infusion pumps may be worn by the user, may use replaceable medicament cartridges, and may deliver other medicaments alone, or in combination with insulin. Such medicaments include glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as continuous glucose monitoring (CGM) devices. A CGM device consists of a sensor placed under the patient's skin and affixed to the patient via an adhesive patch, a transmitter, and a monitor. A CGM device samples the patient's interstitial fluid periodically (e.g. once every 1-5 minutes) to estimate blood glucose levels over time. CGMs are advantageous because they provide more frequent insights into a user's blood glucose levels yet do not require a finger stick each time a reading is taken.

Ambulatory infusion pumps may incorporate a CGM within the infusion pump device or may communicate with a dedicated CGM directly via a wired connection or indirectly via a wireless connection using wireless data communication protocols to communicate with a separate device (e.g., a dedicated remote device or a smartphone). One example of integration of ambulatory infusion pumps with CGM devices is described in U.S. Patent Publication No. 2014/0276419, which is hereby incorporated by reference herein. Ambulatory infusion pumps typically allow the user or caregiver to adjust the amount of insulin or other medicament delivered by a basal rate or a bolus, based on blood glucose data obtained by a CGM device, and in some cases include the capability to automatically adjust such medicament delivery. For example, based on CGM readings, some ambulatory infusion pumps may automatically adjust or prompt the user to adjust the level of medicament being administered or planned for administration or, in cases of abnormally low blood glucose readings, reducing or temporarily ceasing insulin administration.

In some cases, ambulatory insulin pumps may be configured to deliver insulin based on CGM data in a closed-loop or semi-closed-loop fashion. Some systems including these features may be referred to as automated insulin delivery (AID) systems or artificial pancreas systems because these systems serve to mimic biological functions of the pancreas for persons with diabetes.

The delivery of insulin or other medicament from a portable infusion pump making use of CGM data necessitates accurate and reliable CGM data output. Some CGM devices are calibrated with blood samples to correlate actual blood glucose data with the CGM readings. However, such calibrations are only done periodically, such as every few days or hours (e.g., 12 hours), and the longer it has been since a calibration event the more likely the CGM is unreliable to some degree and the more unreliable the CGM is likely to become until the next calibration. In addition, because of the need for wireless connectivity with the CGM, any failure of the CGM sensor, loss of signal or communication between the CGM and the pump, etc., can cause the algorithm calculating therapy doses to not receive the CGM readings. Automatically dosing medicaments such as insulin based on CGM readings can have potentially dangerous effects in situations where the CGM readings are inaccurate or unreliable relative to the user's actual blood glucose levels, or where the CGM readings are not received, due to over-delivery or under-delivery of insulin. Existing systems therefore generally stop automated delivery when the CGM readings are known to be inaccurate or where the readings are not received. In addition, a user may be able to manually turn off a closed loop control feature.

When automated delivery is ceased, such systems revert to open loop therapy that is not based on CGM data. Typically, upon initiating open loop therapy the system will deliver a pre-programmed basal rate stored in memory.

### SUMMARY

Disclosed herein are apparatuses and methods for transitioning from automated closed loop insulin delivery to open loop insulin therapy. When closed loop control is terminated, rather than immediately transitioning from the closed loop rate at termination to a preprogrammed open loop rate, the system can instead gradually transition over time to the preprogrammed open loop rate. This gradual transition provides a safer transition to open loop therapy that reduces the risk of hyperglycemia and hypoglycemia.

In an embodiment, an ambulatory infusion pump system can include a pump mechanism configured to facilitate delivery of a medicament to a patient, a memory adapted to store an open-loop basal rate profile for the patient, a communications interface adapted to receive glucose levels from a continuous glucose monitor and at least one processor. The at least one processor can be configured to cause the pump mechanism to deliver the medicament to the patient in a closed-loop mode in which therapy parameters are automatically determined and medicament is automatically delivered according to the therapy parameters based on the glucose levels from the continuous glucose monitor. When the closed loop mode is terminated, the at least one processor can gradually transition from the therapy parameters from the closed-loop mode to the open-loop basal rate profile stored in memory.

In an embodiment, a method of diabetes therapy can include storing an open-loop basal rate profile for a patient, receiving glucose levels from a continuous glucose monitor and delivering medicament to the patient with an infusion pump in a closed-loop mode in which therapy parameters are automatically determined and medicament is automatically delivered according to the therapy parameters based on the glucose levels from the continuous glucose monitor. Upon terminating closed loop mode the method can gradually transition from the therapy parameters from the closed-loop mode to the open-loop basal rate profile stored in memory.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention may be more completely understood in consideration of the following detailed description of various embodiments of the invention in connection with the accompanying drawings, in which:

FIG. 1 is an embodiment of an ambulatory infusion pump for use with embodiments of the disclosure.

FIG. 2 is a block diagram of the ambulatory infusion pump of FIG. 1. FIGS. 3A-3B are an alternate embodiment of an ambulatory infusion pump for use with embodiments of the disclosure.

FIG. 4 is an embodiment of a CGM for use with embodiments of the disclosure.

FIGS. 5A-5B depict delivery profiles for delivery of medicament with an infusion pump according to the disclosure.

FIG. 6 is a flowchart of method steps in a method for diabetes therapy according to the disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

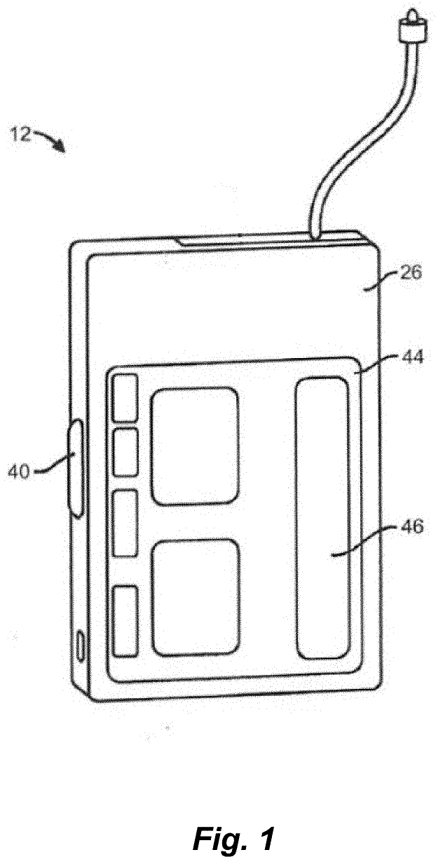

FIG. 1 depicts an example infusion pump that can be used in conjunction with one or more embodiments of the ambulatory infusion pump system of the present disclosure. Pump 12 includes a pumping or delivery mechanism and reservoir for delivering insulin or other medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display (e.g., a dedicated remote display or a CGM display), a remote control device, or a consumer electronic device (e.g., laptop computer, personal computer, tablet computer, smartphone, electronic watch, electronic health or fitness monitor, or personal digital assistant). Further details regarding such pump devices can be found in U.S. Pat. No. 8,287,495, previously incorporated by reference above. It is to be appreciated that pump 12 may be optionally configured to deliver one or more additional or other medicaments to a patient.

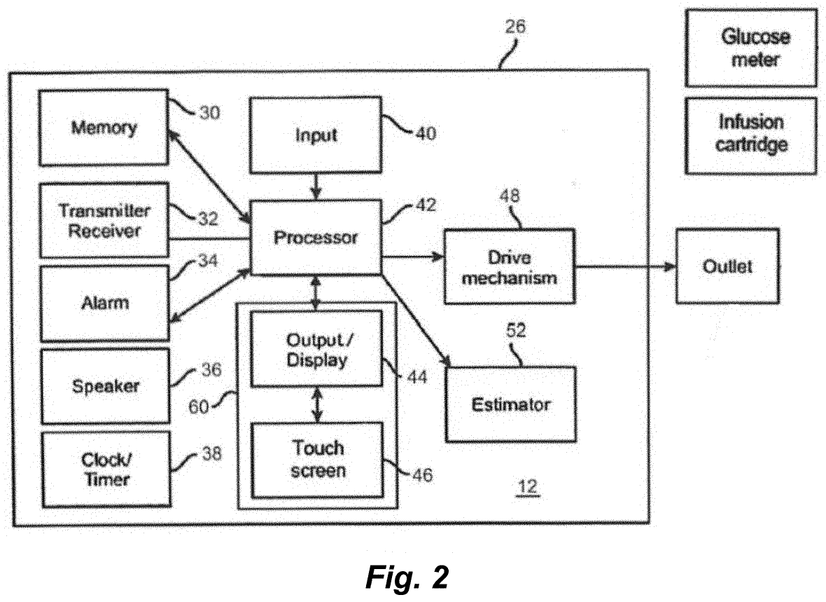

FIG. 2 illustrates a block diagram of some of the features that may be included within the housing 26 of pump 12. The pump 12 can include a processor 42 that controls the overall functions of the pump. The pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices through the transmitter/receiver 32 such as a remote device (e.g., CGM device), a remote control device, or a consumer electronic device (e.g., laptop computer, personal computer, tablet computer, smartphone, electronic watch, electronic health or fitness monitor, or personal digital assistant). In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. The processor 42 may also include programming to receive signals and/or other data from an input device, such as, by way of example, a pressure sensor, a temperature sensor, or the like.

FIGS. 3A-3B depicts a second infusion pump that can be used in conjunction with one or more embodiments of the ambulatory infusion pump system of the present disclosure. Pump 102 includes a pump drive unit 118 and a medicament cartridge 116. Pump 102 includes a processor that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receive commands from a separate device for control of some or all of the operations of the pump. Such separate device can include, for example, a dedicated remote control device or a consumer electronic device such as a smartphone having a processor executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Pat. No. 10,279,106 and U.S. Patent Publication Nos. 2016/0339172 and 2017/0049957, each of which is hereby incorporated herein by reference in its entirety.

In some embodiments, all elements of an infusion pump system such as, e.g., the user interface, processor(s), pump mechanism, etc., reside in a single device, such as an infusion pump. In other embodiments, an infusion pump system may be a distributed system in which portions of the functionality such as, e.g., the user interface, speaker, processor, dosing algorithm, etc. may reside in separate devices such as in the infusion pump, dedicated remote control and/or other mobile device such as a mobile phone, or central computer system such as a cloud computing system.

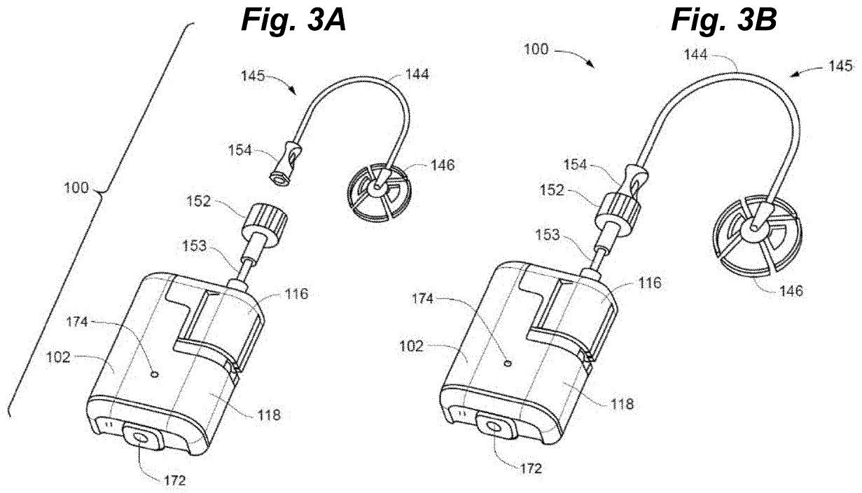

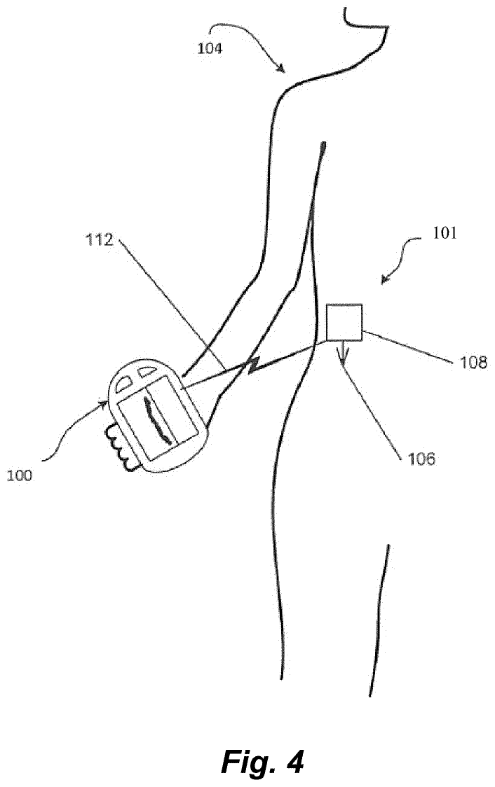

FIG. 4 depicts an example CGM system that can be used in conjunction with one or more embodiments of the ambulatory infusion pump system of the present disclosure. The CGM system includes a sensor 101, a sensor probe 106, a sensor body 108, a receiver, and a monitor (receiver and monitor are depicted as device 100 in FIG. 4). The sensor 101 is removably affixed to a user104 and includes a sensor probe 106 configured for transcutaneous insertion into the user 104. When placed, the sensor probe 106 reacts with the user's interstitial fluid which produces a signal that can be associated with the user's blood glucose level. The sensor 101 further includes a sensor body 108 that transmits data associated with the signal to the receiver 100 via wired or wireless connection (as represented by arrow line 112). In preferred embodiments, the receiver 100 receives the transmitted data wirelessly by any suitable means of wireless communication. By way of example only, this wireless communication may include a near field communication (NFC) radio frequency (RF) transmitter or a transmitter operating according to a “Wi-Fi” or Bluetooth® protocol, Bluetooth® low energy protocol or the like. Further detail regarding such systems and definitions of related terms can be found in, e.g., U.S. Pat. Nos. 8,311,749, 7,711,402 and 7,497,827, each of which is hereby incorporated by reference in its entirety.

With the infusion pump and CGM interfaced, the CGM can automatically transmit the CGM data to the pump. The pump can then use this data to automatically determine therapy parameters and suggest a therapy adjustment to the user or automatically deliver the therapy adjustment to the user. These therapy parameters including thresholds and target values can be stored in memory located in the pump or, if not located in the pump, stored in a separate location and accessible by the pump processor (e.g., “cloud” storage, a smartphone, a CGM, a dedicated controller, a computer, etc., any of which is accessible via a network connection). The pump processor can periodically and/or continually execute instructions for a checking function that accesses these data in memory, compares them with data received from the CGM and acts accordingly to adjust therapy. In further embodiments, rather than the pump determining the therapy parameters, the parameters can be determined by a separate device and transmitted to the pump for execution. In such embodiments, a separate device such as the CGM or a device in communication with the CGM, such as, for example, a smartphone, dedicated controller, electronic tablet, computer, etc. can include a processor programmed to calculate therapy parameters based on the CGM data that then instruct the pump to provide therapy according to the calculated parameters.

For example, if the CGM readings indicate that the user has or is predicted to have a high blood glucose level, the ambulatory infusion system can automatically calculate an insulin dose sufficient to reduce the user's blood glucose level below a threshold level or to a target level and automatically deliver the dose. Alternatively, the ambulatory infusion system can automatically suggest a change in therapy upon receiving the CGM readings such as an increased insulin basal rate or delivery of a bolus, but can require the user to accept the suggested change prior to delivery rather than automatically delivering the therapy adjustments.

By way of further example, if the CGM readings indicate that the user has or is predicted to have a low blood glucose level (hypoglycemia), the ambulatory infusion system can, for example, automatically reduce or suspend a basal rate, suggest to the user to reduce a basal rate, automatically deliver or suggest that the user initiate the delivery of an amount of a substance such as, e.g., a hormone (glucagon) to raise the concentration of glucose in the blood, automatically suggest that the patient address the hypoglycemic condition as necessary (e.g., ingest carbohydrates), singly or in any desired combination or sequence.

Automated insulin delivery (AID) systems such as those described above require accurate and reliable glucose values from the CGM and therefore such systems typically terminate automated delivery for safety of the patient when the system determines that the CGM data is inaccurate or unreliable or when connectivity or other issues stop the algorithm from receiving the CGM values. Such systems can also generally be manually terminated by a user by turning off the closed loop delivery mode. When closed loop mode is terminated, current systems abruptly return immediately to the user's preprogrammed basal rate. However, this abrupt transition can be problematic. For example, some users may have outdated basal rates and may therefore get more or less insulin than they actually need from the preprogrammed rate. In addition, the preprogrammed rate could lead to hypoglycemia for users who are at a low glucose level and/or have had insulin delivery suspended due to a low glucose level at the time that closed loop therapy is terminated or to hyperglycemia for user who are at a high glucose level at the time that closed loop therapy is terminated. Embodiments disclosed herein provide for a safer transition from closed loop mode to open loop mode than abruptly transitioning to a preprogrammed rate.

In embodiments, systems and methods disclosed herein can gradually return delivery to the preprogrammed rate rather than immediately returning to the preprogrammed rate. Such a system provides an important safeguard against hypoglycemia and hyperglycemia if the user is in one of the circumstances noted above. By returning to the preprogrammed rate more slowly, the user is provided with the advantages both of potentially being able to resume closed loop therapy prior to reaching the preprogrammed rate and of being able to feel if the therapy is not meeting the user's bodily needs prior to reaching the preprogrammed rate.

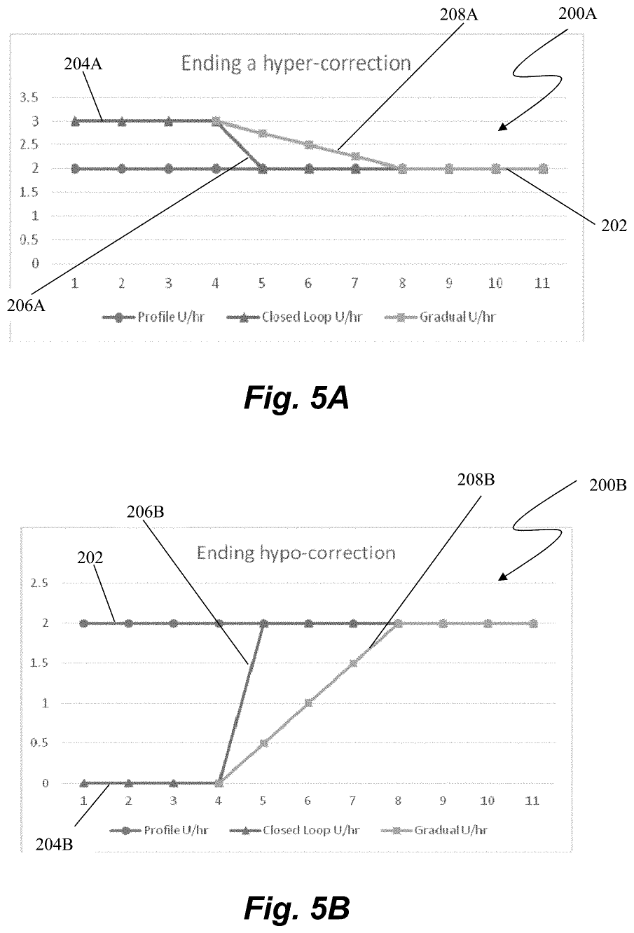

Referring to FIGS. 5A-5B, delivery profiles for a gradual return to a preprogrammed rate are depicted. FIG. 5A depicts a hyperglycemic correction 200A, i.e., when insulin delivery was increased at the time closed loop therapy was terminated to address high glucose levels and FIG. 5B depicts a hypoglycemic correction 200B, i.e., when insulin delivery was reduced or suspended at the time closed loop therapy was terminated to address low glucose levels. In both figures, closed loop control is terminated at interval 5 and the pump returns to a preprogrammed rate 202 of 2 units/hr.

Referring to FIG. 5A, prior to termination of the closed loop mode the pump was delivering a closed loop hyper-correction rate 204A greater than the preprogrammed rate 202 of 3 units/hr. Such an increased, hyper-correction rate would be delivered, for example, if the user's glucose levels were high and/or increasing at a certain rate. After termination of the closed-loop mode at interval 5, rather than providing an immediate return 206A to the pre-programmed rate 202, the gradual return rate 208A gradually returns to the preprogrammed rate 202 over the course of several time intervals.

In FIG. 5B, prior to termination of the closed loop mode the pump was delivering a closed loop hypo-correction 204B less than the preprogrammed rate 202 of 0 u/hr (i.e., basal delivery was suspended). Such a reduced or suspended delivery rate would be delivered, for example, if the user's glucose levels were low and/or decreasing at a certain rate. After termination of the closed-loop mode at interval 5, rather than immediately returning to the preprogrammed rate 202 at 206B, a gradual return rate 208B over the course of several time intervals as applied to gradually return the user to the preprogrammed rate 202.

In the embodiments depicted in FIGS. 5A-5B, the gradual return rates 208A, 208B gradually return to the preprogrammed open loop rate 202 over a time period of four closed loop time intervals, but the time over which the rate returns to the preprogrammed rate can be longer or shorter and can be a predetermined, fixed amount of time or intervals or can vary. In embodiments, the speed with which the system returns to the open loop rate can be based on a risk of hyperglycemia and/or hypoglycemia determined based on one or more of several factors for the specific termination event. Factors that can be taken into account in determining the risk of hypo/hyperglycemia can include, for example, one or more of a most recent CGM value prior to termination, a most recent CGM trend at termination, an amount of time over which the system had been delivering increased or decreased insulin amounts and/or future glucose predictions. For example, if the most recent CGM value or future glucose prediction is within a target range, the most recent CGM trend showed relatively stable glucose levels and/or the system had not been delivering or had only been delivering increased or decreased insulin amounts for a short period of time, the system may determine that the risk of hypo/hyperglycemia is low. In such a circumstance, the system can return to the preprogrammed rate relatively quickly. Conversely, if the most recent CGM value or future glucose prediction is above or below a high or low glucose threshold, the most recent CGM trend showed increasing or decreasing glucose levels at a rate over a predetermined rate, or the system had been delivering increased or decreased insulin levels for an extended period of time at termination, the system may determine that the risk of hypo/hyperglycemia is high and return to the preprogrammed rate over a longer period of time. Such determinations can also be made based on a combination of these factors. For example, if the most recent CGM value is not over the predetermined high threshold but is increasing at a certain rate, or if the value is not below the predetermined low threshold but is decreasing at a certain rate, the system can return more slowly. Similarly, if the CGM value is over a high or low threshold but is decreasing or increasing at a certain rate such that the user's blood glucose level is likely to cross the threshold into a safe range in the near future, the system can return more quickly.

In addition, although FIGS. 5A-5B depict that the gradual return occurs at a constant rate (e.g., 0.5 units/interval for the gradual rate 208B in FIG. 5B), the rate of the gradual return can alternatively vary over time. For example, the rate can initially return towards the preprogrammed rate in smaller increments that increase over time such that the gradual rate returns to the preprogrammed rate more rapidly over time.

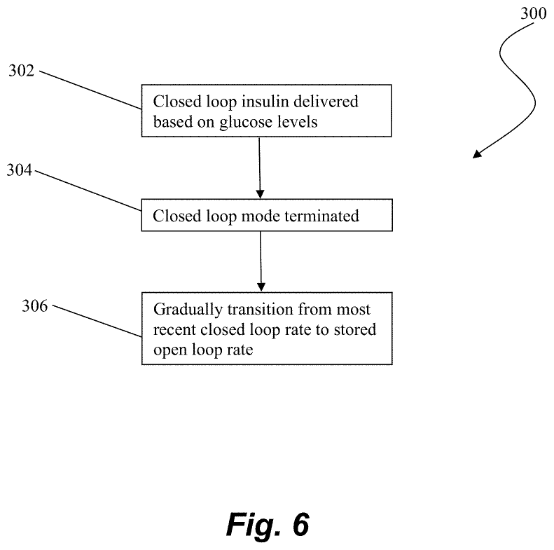

Referring now to FIG. 6, a flowchart of method steps in a method of diabetes therapy 300 according to the disclosure is depicted. At step 302, insulin and/or other medicaments are automatically being delivered to a user with a closed loop algorithm based on glucose levels of the user. Closed loop mode is terminated at step 304. Closed loop mode can be terminated for a variety of reasons, including, for example, connectivity issues with the CGM or other accuracy or reliability issues with the CGM, manually by the user, etc. At step 306, the system gradually transitions the delivery rates for the user from the most recent closed loop rate to a stored open loop rate. As discussed above, this gradually transition can occur at varying speeds and/or in varying magnitudes based on a variety of factors.

As noted above, the gradual return rates disclosed herein provide for a safer transition from closed loop mode to open loop mode than abruptly transitioning to a preprogrammed rate to reduce the risk of hyperglycemia and hypoglycemia. A gradual return to the preprogrammed rate could provide patient benefit particularly when the system has terminated closed loop control due to poor CGM connectivity by not transitioning too far from the closed loop rate too quickly in the event that connectivity is reestablished.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing

### DETAILED DESCRIPTION

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; and 11,224,693 and commonly owned U.S. Patent Publication Nos. 2009/0287180; 2012/0123230; 2013/0053816; 2014/0276423; 2014/0276569; 2014/0276570; 2018/0071454; 2019/0240398; 2019/0307952; 2020/0206420; 2020/0261649; 2020/0306445; 2020/0329433; 2020/0368430; 2020/0372995; 2021/0001044; 2021/0113766; 2021/0154405; and 2021/0353857 and commonly owned U.S. patent applications Ser. Nos. 17/368,968; 17/459,129; 17/517,885; 17/573,705; 17/587,412; 17/587,434 and 17/587,468.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An ambulatory infusion pump system, comprising:
a pump mechanism configured to facilitate delivery of a medicament to a patient
a memory adapted to store an open-loop basal rate profile for the patient;
a communications interface adapted to receive glucose levels from a continuous glucose monitor; and
at least one processor configured to:
cause the pump mechanism to deliver the medicament to the patient in a closed-loop mode in which therapy parameters are automatically determined and medicament is automatically delivered according to the therapy parameters based on the glucose levels from the continuous glucose monitor;
terminate the closed-loop mode; and
gradually transition from the therapy parameters from the closed-loop mode to the open-loop basal rate profile stored in memory upon terminating closed loop mode.

2. The ambulatory infusion pump system of claim 1, wherein the gradual transition is linear.

3. The ambulatory infusion pump system of claim 1, wherein the gradual transition is non-linear.

4. The ambulatory infusion pump system of claim 1, wherein the processor is further configured to determine a risk of hyperglycemia or hypoglycemia from the open-loop basal rate profile and a speed for the gradual transition is based on the risk.

5. The ambulatory infusion pump system of claim 4, wherein the speed for the gradual transition is greater if there is a low risk of both hyperglycemia and hypoglycemia.

6. The ambulatory infusion pump system of claim 4, wherein the processor is configured to determine the risk of hyperglycemia or hypoglycemia based on one or more of a most recent glucose level from the continuous glucose monitor, a most recent glucose level trend based on the glucose levels from the continuous glucose monitor, a future glucose level prediction and whether and how long the closed-loop made was delivering medicament greater or lower than the open-loop basal rate profile.

7. The ambulatory infusion pump system of claim 4, wherein it is determined that the risk of hyperglycemia and hypoglycemia is low if the user's glucose levels are within a target glucose range.

8. The ambulatory infusion pump system of claim 1, wherein closed loop mode is terminated if the communications interface is not receiving glucose levels from the continuous glucose monitor.

9. The ambulatory infusion pump system of claim 1, wherein closed loop mode is terminated if it is determined the glucose levels from the continuous glucose monitor may be inaccurate or unreliable.

10. The ambulatory infusion pump system of claim 1, wherein closed loop mode is manually terminated by the patient.

11. A method of diabetes therapy, comprising:
storing an open-loop basal rate profile for a patient;
receiving glucose levels from a continuous glucose monitor; and
delivering medicament to the patient with an infusion pump in a closed-loop mode in which therapy parameters are automatically determined and medicament is automatically delivered according to the therapy parameters based on the glucose levels from the continuous glucose monitor;
terminating closed loop mode; and
gradually transitioning from the therapy parameters from the closed-loop mode to the open-loop basal rate profile stored in memory upon terminating closed loop mode.

12. The method of claim 11, wherein gradually transitioning includes linearly transitioning to the open loop basal rate profile.

13. The method of claim 1, wherein gradually transitioning includes a non-linear transition to the open loop basal rate profile.

14. The method of claim 11, further comprising determining a risk of hyperglycemia or hypoglycemia from the open-loop basal rate profile and a speed for the gradual transition is based on the risk.

15. The method of claim 14, wherein the speed for the gradual transition is greater if there is a low risk of both hyperglycemia and hypoglycemia.

16. The method of claim 14, wherein determining the risk of hyperglycemia or hypoglycemia includes using one or more of a most recent glucose level from the continuous glucose monitor, a most recent glucose level trend based on the glucose levels from the continuous glucose monitor, a future glucose level prediction and whether and how long the closed-loop made was delivering medicament greater or lower than the open-loop basal rate profile.

17. The method of claim 14, wherein it is determined that the risk of hyperglycemia and hypoglycemia is low if the user's glucose levels are within a target glucose range.

18. The method of claim 11, wherein closed loop mode is terminated if glucose levels are not being received from the continuous glucose monitor.

19. The method of claim 11, wherein closed loop mode is terminated if it is determined the glucose levels from the continuous glucose monitor may be inaccurate or unreliable.

20. The method of claim 11, wherein closed loop mode is manually terminated by the patient.
